(19) United States
(12) Patent Application Publication
(10) Pub. No.: US 2016/0045356 A1
(43) Pub. Date: Feb. 18, 2016

(54) Medical Compression Device for Animals

(71) Applicants: Neil Grossman, Arcata, CA (US); Sari L Swede, Arcata, CA (US)

(72) Inventors: Neil Grossman, Arcata, CA (US); Sari L Swede, Arcata, CA (US)

(21) Appl. No.: 14/827,161

(22) Filed: Aug. 14, 2015

Related U.S. Application Data
(60) Provisional application No. 62/037,507, filed on Aug. 14, 2014.

(51) Int. Cl. A61F 5/32; A61F 13/14; A61F 13/12; A61F 13/00

(52) U.S. Cl. CPC A61F 5/32; A61F 13/00017; A61F 13/00038; A61F 13/00059; A61F 13/00068; A61F 13/146; A61F 13/143; A61F 13/148; A61F 13/128

(57) ABSTRACT

A compression garment designed for use on the neck, chest, and torso of a four-legged animal. The garment has adjustable sections that can be calibrated to specific levels of compression using tensioning straps. By setting a specific part of the compression garment to a specific pressure level, surgical sites, wounds, and other injuries can be treated by reducing edema and increasing circulation in the specific part of the animal treated by the calibrated, compressed section of the garment. Some embodiments of the invention additionally use the PowerNet fabric to achieve uniform compression and/or accommodate a Jackson-Pratt drain to facilitate active draining of care sites.

### CROSS-REFERENCE TO RELATED APPLICATION

This application claims the benefit under 35 U.S.C. §119(e) of U.S. Provisional Application No. 62/037507 filed Aug. 14, 2014, which is incorporated herein by reference.

### BACKGROUND OF THE INVENTION

Compression garments have long been used to treat circulatory-related ailments such as diabetes, deep vein thrombosis, and edema. More recently, compression clothing has been used by athletes to enhance performance by increasing blood flow to muscles and reducing swelling and soreness. Both medical and athletic applications of compression garments work by redirecting blood flow and fluids to reduce swelling and increase circulation.

In animal applications, compression garments have typically been used on the extremities to reduce swelling of the extremities. These garments have also been used on the torso to calm the animal, similar to the use of squeeze chutes to calm cattle. However, an additional problem that has received less attention but can be treated by compression therapy is the build-up of fluid around wounds or surgical sites. This edema can not only slow recovery but can also cause inflammation, infection, and necrosis of the surrounding tissue. When necrosis occurs, the dead tissue must be removed through additional surgery. This additional surgery not only does not solve the original problem of edema and potential necrosis, but it also carries additional risks and potential side-effects during and after the surgery, especially if the surgery is conducted under general anesthesia.

Often, bandages have been used to attempt to apply pressure to wounds and surgery sites to prevent edema and its attendant problems. However, it is difficult to apply any substantial pressure to the wound site using standard bandages. Additionally, existing bandages do not allow a specific and adjustable amount of pressure to be selected for use upon the care site.

### SUMMARY OF THE INVENTION

The present invention is a compression garment designed for use on the neck, chest, and torso of a four-legged animal. The garment has adjustable sections that can be calibrated to specific levels of compression using tensioning straps. By setting a specific part of the compression garment to a specific pressure level, surgical sites, wounds, and other injuries can be treated by reducing edema and increasing circulation in the specific part of the animal treated by the calibrated, compressed section of the garment. Some embodiments of the invention additionally use the PowerNet fabric to achieve uniform compression and/or accommodate a Jackson-Pratt drain to facilitate active draining of care sites.

### BRIEF DESCRIPTION OF THE DRAWINGS

FIG. 1 is an orthogonal view of the top side of a compression garment when the garment is placed on a flat surface.

FIG. 2 is an orthogonal view of the bottom side of the compression garment of FIG. 1 when the garment is placed on a flat surface.

FIG. 3 is a perspective view of the compression garment of FIG. 1 showing the first step of placing the compression garment on an exemplary four-legged animal.

FIG. 4 is a perspective view of the compression garment of FIG. 1 showing the second step of placing the compression garment on an exemplary four-legged animal.

FIG. 5 is a perspective view of the compression garment of FIG. 1 showing the third step of placing the compression garment on an exemplary four-legged animal.

FIG. 6 is a perspective view of the compression garment of FIG. 1 showing the fourth step of placing the compression garment on an exemplary four-legged animal.

FIG. 7 is an orthogonal view of part of the right side of the compression garment of FIG. 1 showing the first step of pulling the tensioning strap to the desired pressure level.

FIG. 8 is an orthogonal view of part of the right side of the compression garment of FIG. 1 showing the second step of pulling the tensioning strap to the desired pressure level.

FIG. 9 is an orthogonal view of part of the right side of the compression garment of FIG. 1 showing the third step of pulling the tensioning strap to the desired pressure level.

FIG. 10 is a perspective front view of an exemplary animal wearing the compression garment of FIG. 1.

FIG. 11 is an orthogonal left side view of an exemplary animal wearing the compression garment of FIG. 1.

FIG. 12 is an orthogonal right side view of an exemplary animal wearing the compression garment of FIG. 1.

FIG. 13 is an orthogonal view of the top side of a compression garment when the garment is placed on a flat surface, wherein the garment contains an opening and pocket for securing the tubing and bulb portions of a Jackson-Pratt drain.

FIG. 14 is an orthogonal right side view of an exemplary animal wearing the compression garment of FIG. 13.

REFERENCE NUMERALS

100 Compression garment

101 Top side of compression garment

102 Top section

104 Left bottom section

106 Right bottom section

108 Central section

110 Strips containing loop portion of hook-and-loop attachments

112 Tensioning straps

114 Tensioning strap loops

116 Left bottom section fastener strap

118 Right bottom section fastener strap

120 Left bottom section male buckle

122 Right bottom section female buckle

124 Adjustment clips

126 Right bottom section third fastener strap

128 Right bottom section third fastener strap male buckle

130 Right bottom section third fastener strap female buckle

132 Gap in middle of right bottom section

134 Bottom side of compression garment

136 Strips containing hook portion of hook-and-loop attachments

138 Exemplary four-legged animal

140 Loop for drain tubing

142 Pocket for drain bulb

### DETAILED DESCRIPTION OF THE INVENTION

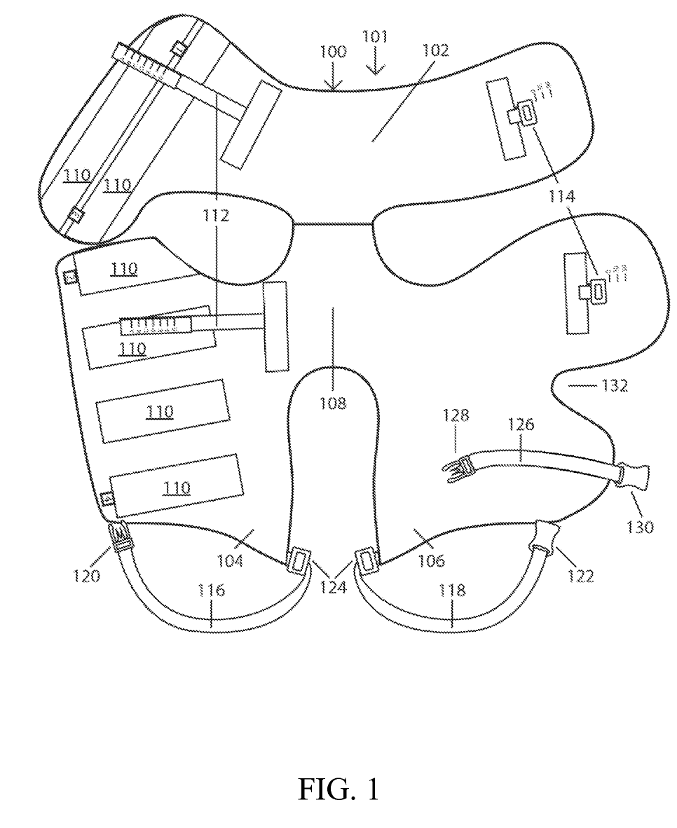

Referring now to FIG. 1, the top side 101 of a compression garment 100 is shown. The garment 100 is intended for use on an animal having a central elongated torso, a neck, and four limbs organized into two pairs, with the first pair of limbs descending from the front of the torso and the second pair of limbs descending from the rear of the torso. The compression garment 100 has a tri-part structure bisected by a horizontal axis into an elongated top section 102 and two bottom sections 104, 106. Each of the bottom sections 104, 106 is partially separated from the other bottom section by a gap between the ends of the bottom sections 104, 106 that are distal to the horizontal axis. Each of the bottom sections 104, 106 is connected to the other bottom section at the end of the bottom section that is proximal to the horizontal axis. This central section 108 connecting the two bottom sections is also connected to the horizontal central portion of the elongated top section 102 that is proximal to the horizontal axis. The garment 100 has multiple strips of the loop part 110 of hook-and-loop attachments on the top side 101 of the garment 100 located on both the left portion of the top section 102 and the left bottom section 104. The left portion of the top section 102 and the left bottom section 104 both also contain tensioning straps 112 that are designed to fit into loops 114 located on the right portion of the top section 102 and the right bottom section 106. Each of these tensioning straps 112 has written on it the letters A through G, and to the right of each letter is a horizontal dash mark. The opposite side of each tensioning strap 112 that does not contain any letters or markings has two halves of a hook-and-loop fastener attached to it; one half of the fastener is attached to the proximal end of the strap 112 and the other half of the fastener is connected to the distal end of the strap 112. Beneath and to the right of the loops 114 that each tensioning strap is to fit into are three numbers, and to the left of each number is a horizontal dash mark. In the present embodiment of the garment 100, the three numbers are 0, 30, and 50, but other embodiments use different numbers instead of 30 and 50. At the end of each bottom section 104, 106 of the garment 100 that is distal to the horizontal axis is a strap 116, 118 with one half of a buckle 120, 122, which is a male buckle end 120 on the strap 116 attached to the left bottom section 104 and a female buckle end 122 on the strap 118 attached to the right bottom section 106. These bottom section straps 116, 118 are adjustable in length through the movement of an adjustment clip 124 located on each strap that lengthens or shortens the strap 116, 118 when the clip 124 is moved. A third strap 126 is located on the right bottom section 106 of the garment 100. This third strap 126 is connected to the right bottom section 106 at the mid-point between the point where the strap 126 at the distal end of the right bottom section 106 is connected to the right bottom section 106 and the centroid of the right bottom section 106. This third strap 126 contains both a male buckle end 128 located at the left end of the strap 126 and a female buckle end 130 located at the right end of the strap 126. In other embodiments of the garment 100, the male and female buckle members 120, 122, 128, 130 may be rotated among the straps 116, 118, 126. In one of these embodiments, the third strap 126 contains two female buckle members and the other two straps 116, 118 contain male buckle members. The right bottom section 106 additionally contains a small gap 132 in the middle of the side of the right bottom section 106 that is distal from the left bottom section 104. This gap 132 allows the top of the end of the right bottom section 106 that is distal from the left bottom section 104 to have some freedom of movement from the bottom of the end of the right bottom section 106 that is distal from the left bottom section 104. This freedom of movement makes it easier for the animal to move through its normal range of motion despite wearing the garment 100.

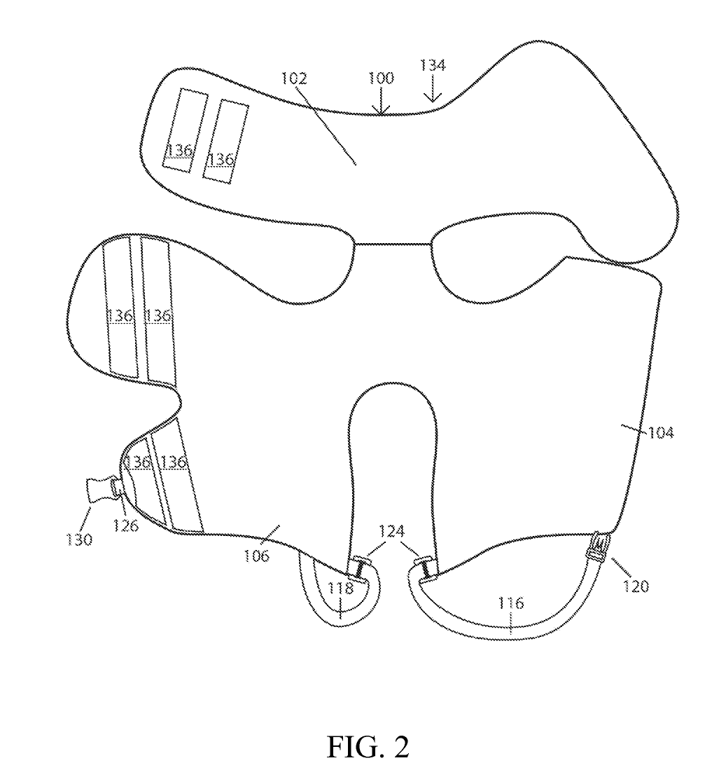

Referring now to FIG. 2, the bottom side 134 of the compression garment 100 is shown. The tri-part structure bisected by a horizontal axis into an elongated top section 102 and two bottom sections 104, 106 is visible from this view. The strap 116, 118 at each end of each bottom section 104, 106 that is distal to the horizontal axis is also visible. The portion of the third strap 126 that contains the female buckle member 130 is also visible. The garment 100 has multiple strips of the hook part 136 of hook-and-loop attachments on the bottom side 134 of the garment 100. These strips 136 are located on both the left portion of the top section 102, which is located opposite of the right portion of the top section 102 of the top side 101 of the garment 100, and the left portion of the bottom view of the right bottom section 106, which is located opposite of the right portion of the right bottom section 106 of the top side 101 of the garment 100.

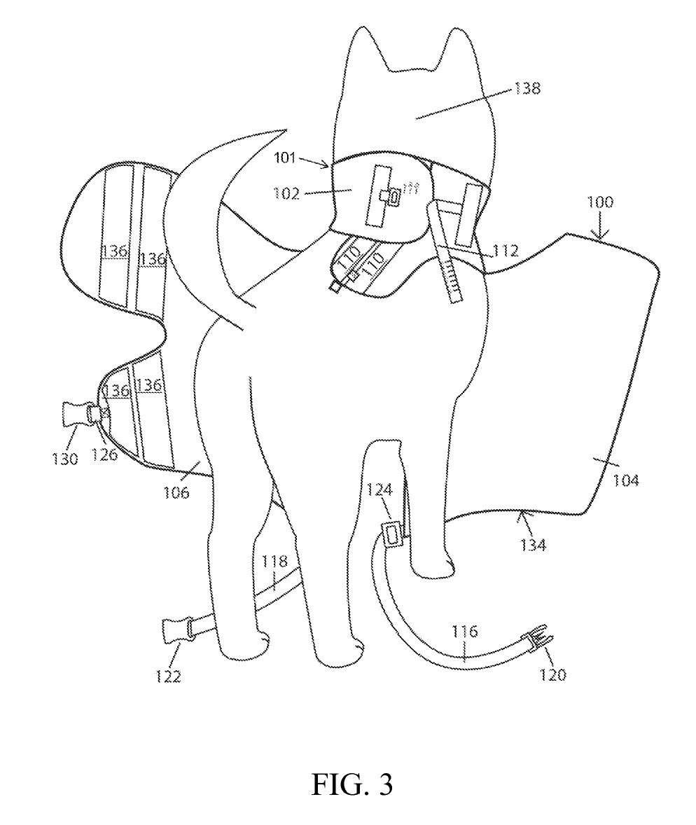

Referring now to FIG. 3, the first step of placing the garment 100 on an exemplary four-legged animal 138 is shown. The top section 102 of the garment 100 is wrapped around the neck of the animal 138, with the left portion of the top section 102 of the top side 101 covered by the right portion of the top section 102, and the two portions being secured against each other by the loop part 110 of hook-and-loop attachments located on the left portion being connected with the hook part 136 of hook-and-loop attachments located on the part of the bottom side of the garment 100 that is opposite the right portion of the top section 102 of the top side 101.

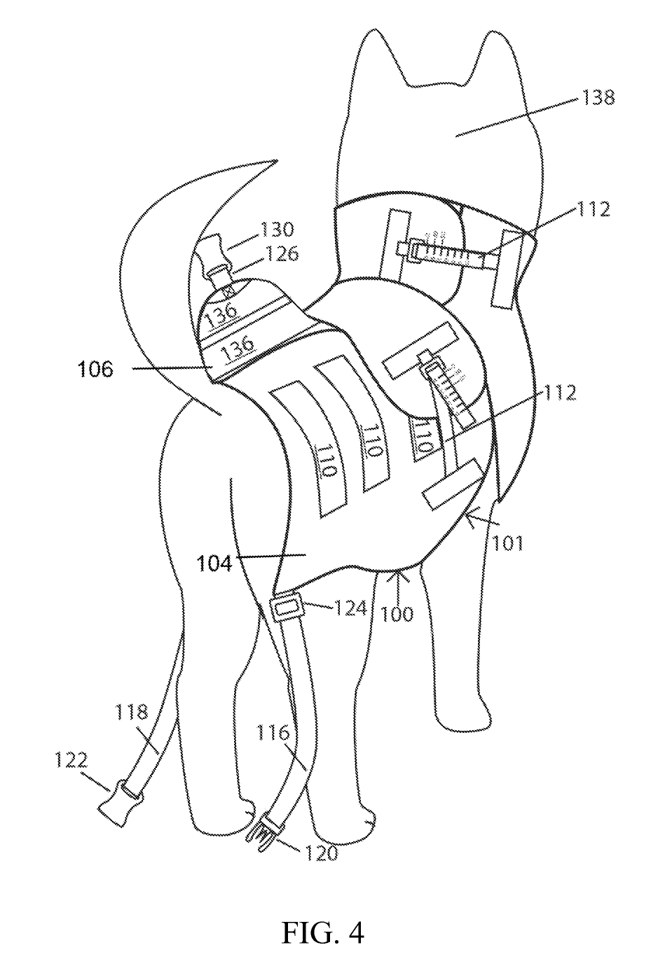

Referring now to FIG. 4, the second step of placing the garment 100 on an exemplary four-legged animal 138 is shown. The left bottom section 104 is placed over the back and right side of the animal 138. The right bottom section 106 is then wrapped around the left side of the animal 138. The two bottom sections 104, 106 are then secured together by connecting the loop part 110 of hook-and-loop attachments located on the left bottom section 104 with the hook part 136 of hook-and-loop attachments located on the part of the bottom side 134 of the garment 100 that is opposite from the top of the end of the right bottom section 106 of the top side 101, with the end of the right bottom section 106 being the end that is distal from the left bottom section 104.

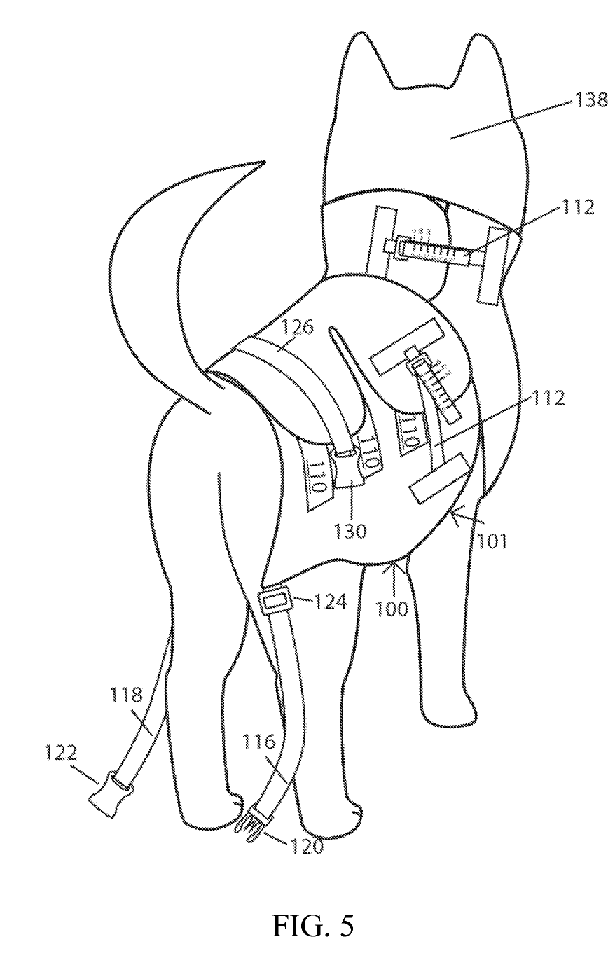

Referring now to FIG. 5, the third step of placing the garment 100 on a four-legged animal 138 is shown. The two bottom sections 104, 106 are further secured together by connecting the loop part 110 of hook-and-loop attachments located on the left bottom section 104 with the hook part 136 of hook-and-loop attachments located on the part of the bottom side 134 of the garment 100 that is opposite from the bottom of the end of the right bottom section 106 of the top side 101, with the end of the right bottom section 106 being the end that is distal from the left bottom section 104. Securing the bottom of the distal end of the right bottom section 106 separately from the top of the distal end of the right bottom section 106 has two advantages. First, the person placing the garment on the animal 138 can create a more secure fit by more easily conforming the garment 100 to the shape of the torso of the animal 138. Second, the animal 138 wearing a garment 100 that is partially split along the back of the torso can more easily move through its normal range of motion despite wearing the garment 100.

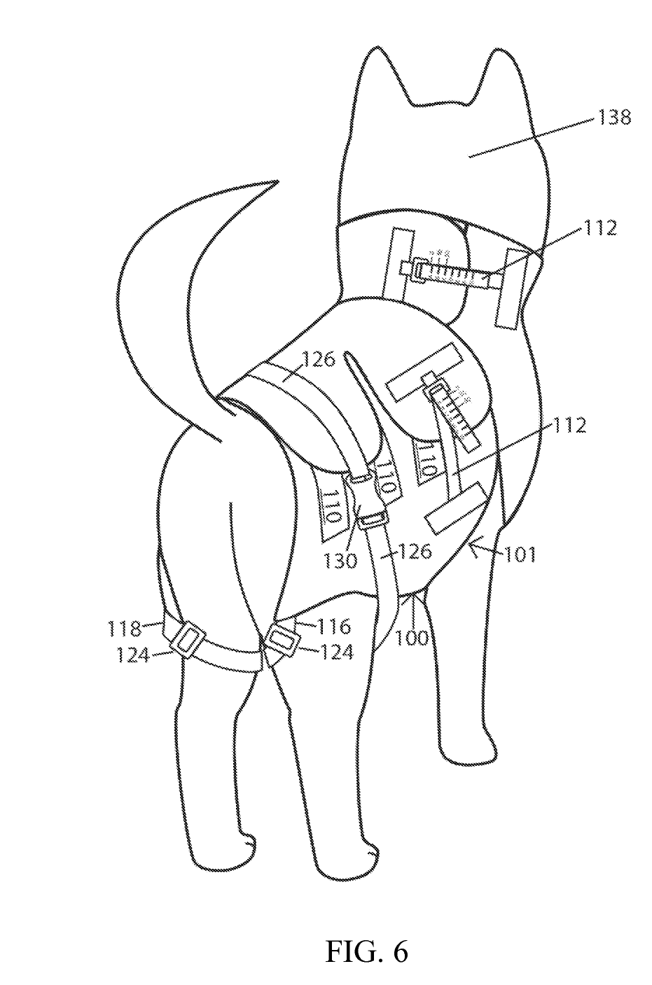

Referring now to FIG. 6, the fourth step of placing the garment 100 on a four-legged animal 138 is shown. The three straps 116, 118, 126 containing buckle end members are fastened around the rear legs of the animal 138, further securing the garment 100. First, the strap 116 connected to the left bottom section 104 is wrapped around the left side of the right hind leg of the animal 138 and up the right side of the animal 138, and the male buckle member 120 at the end of this strap 116 is then connected to the female buckle member 130 on the third strap 126 containing both male and female buckle members 128, 130 located on the right bottom section 106. Next, the strap 118 connected to the right bottom section at the end of the right bottom section that is distal to the horizontal axis is wrapped around the right side of the left hind leg of the animal 138 and up the left side of the animal 138, and the female buckle member 122 connected to this strap 118 is then connected to the male buckle member 128 on the third strap 126 containing both male and female buckle members 128, 130 located on the right bottom section 106. The length of each secured strap 116, 118 can be adjusted through movement of that strap's adjustment clip 124 up or down the strap.

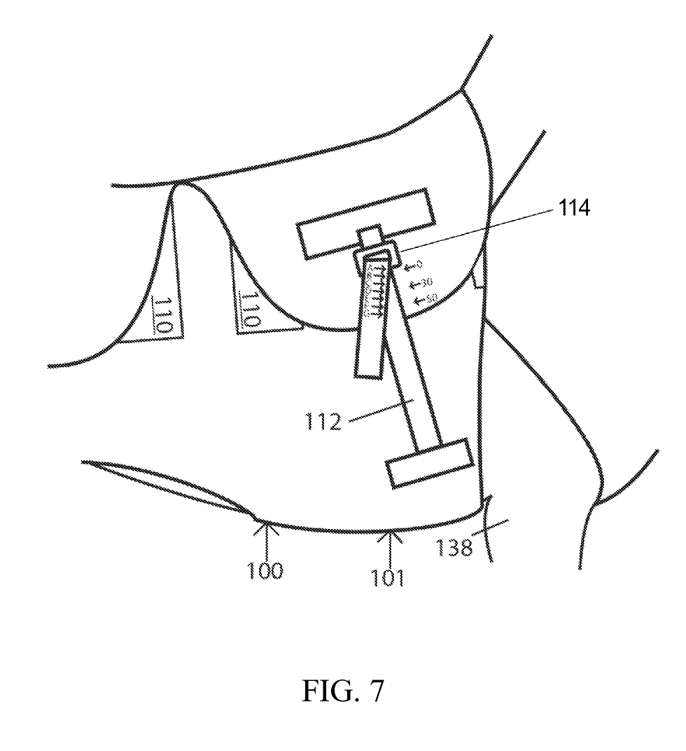

Referring now to FIG. 7, the garment 100 can be adjusted to specific levels of compression through use of pre-calibrated pressure markings The garment 100 has a marking system written on and next to the tensioning straps 112 on the garment 100 that can be used to set the garment 100 to specific levels of compression. As seen in FIGS. 1, 4 to 9, and 12 to 14, letters from A through G are written on each of the tensioning straps 112. Next to each letter is a horizontal dash mark. Underneath and to the right of each of the loops 114 through which the tensioning straps 112 are inserted are the numbers 0, 30, and 50, and next to each of these numbers is a horizontal dash mark. Each of the numbers denotes a level of compression measured in mm Hg.

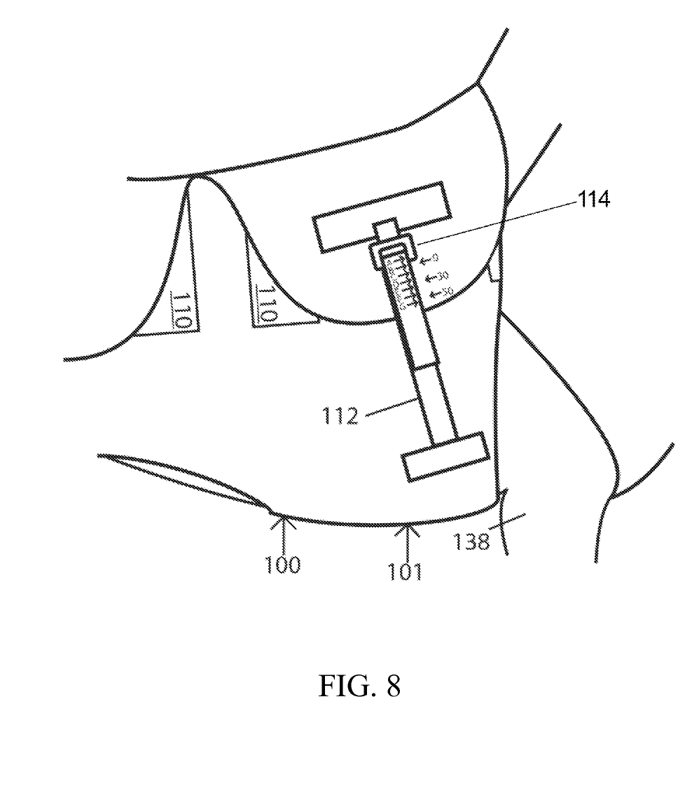

Referring once again to FIG. 7, each tensioning strap 112 is inserted through the loop 114 that is closest to the strap 112 once the garment 100 has been wrapped around an animal 138 as shown in FIGS. 3 to 6. Referring now to FIG. 8, the part of the tensioning strap 112 that has passed through the loop 114 is pulled above and partially covers the part of the tensioning strap 112 that has not passed through the loop 114. The part of the tensioning strap 112 that has passed through the loop 114 is pulled downward until the garment 100 is secure around the torso of the animal 138 with no discernable pressure applied to the animal 138 by the garment 100. The letter associated with the dash mark that lines up with the dash mark next to the number 0 is then noted. The garment is now providing 0 mm Hg of compression to the animal 138 wearing it.

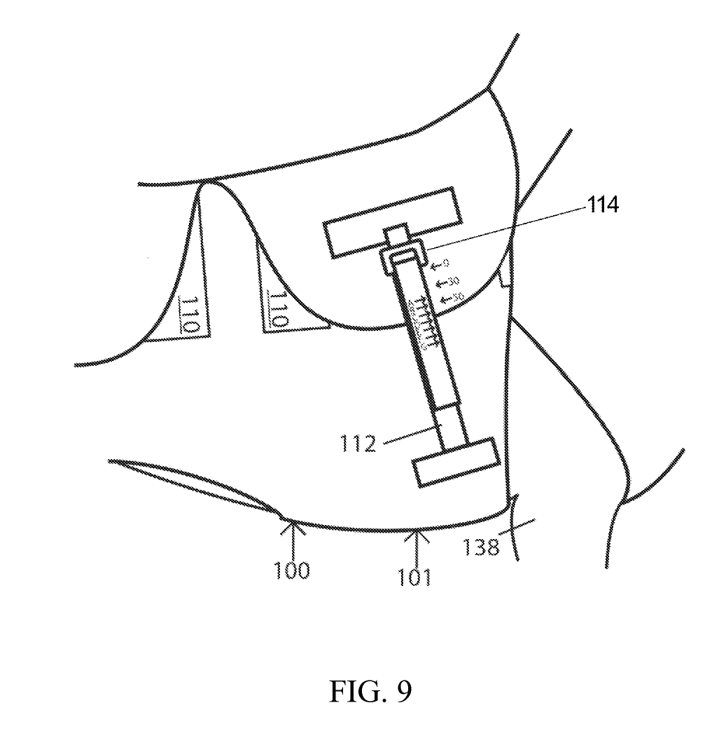

Referring now to FIG. 9, the part of the tensioning strap 112 that has passed through the loop 114 can now be pulled downward to achieve a specific level of compression. When the tensioning strap 112 has been pulled down, as seen in FIG. 9, the letter whose associated dash mark lined up with the dash mark next to the number 0 can be pulled in line with the numbers 30 and 50 through lining up the dash mark of the letter and the dash marks of those numbers. Once the letter is lined up with the number 30, the garment 100 is providing 30 mm Hg compression to the animal 138 wearing it. If the letter is instead lined up with the number 50, the garment 100 will instead provide 50 mm Hg compression to the animal 138 wearing it. Once the desired level of compression is chosen, the tensioning strap 112 can be secured against itself by connecting the two halves of the hook-and-loop fastener located on the side of the tensioning strap 112 opposite from the side on which the letters and markings appear.

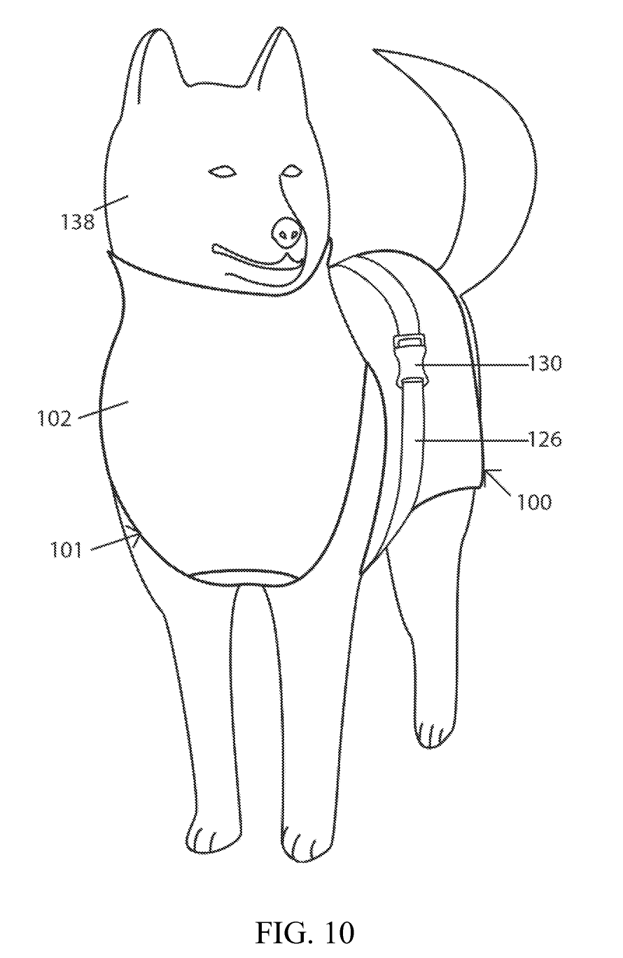

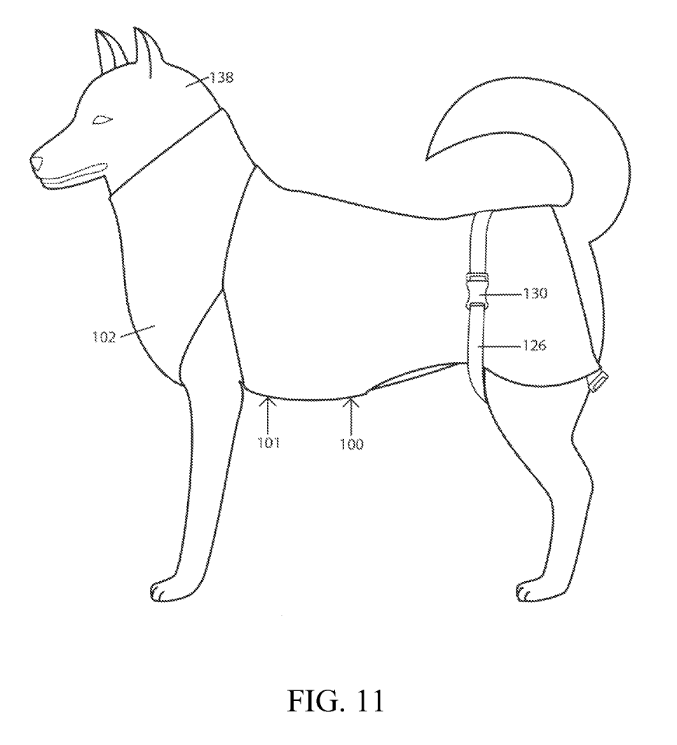

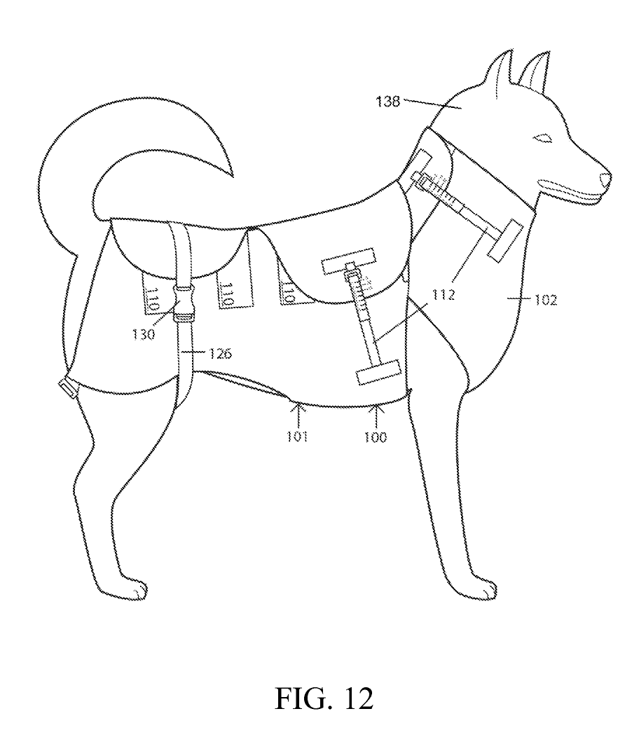

Referring now to FIG. 10, a perspective front view of the garment 100 being worn by a dog is shown. Referring now to FIG. 11, an orthogonal left side view of the garment 100 being worn by a dog is shown. Referring now to FIG. 12, an orthogonal right side view of the garment 110 being worn by a dog is shown.

The present embodiment of the garment 100 comes in a multitude of sizes and fittings that vary based on the species, breed, and size of the animal 138 wearing the garment. For each size of the garment 100, the placement of the 30 and 50 mm Hg numbers and dash marks in relation to the 0 number and dash mark was chosen during the design process based on the compression provided by the garment 100 to an animal 138 that would wear the selected size of the garment 100. A pressure measuring device was placed between the garment 100 and the animal 138 to detect the level of compression based on how far down the tensioning strap 112 is pulled. The numbers 30 and 50, as well as a dash mark next to each number, were placed on the garment 100 at respective distances below the number 0 equal to the distance downward traveled by a given point on the tensioning strap 112 when the compression provided to the model increased from 0 mm Hg to 30 mm Hg and then to 50 mm Hg.

The disclosed embodiment of the garment 100 has two tensioning straps 112 for tightening the garment 100 at the neck and upper torso. However, other embodiments additionally have a tensioning strap for tightening the garment at the lower torso or have material covering the limbs and tensioning straps for adjusting the level of compression around the limbs.

The primary purpose of the compression garment 100 is to provide medically-useful levels of compression to specific areas of an animal 138 wearing the garment 100. By setting a specific part of the compression garment 100 to a specific pressure level, surgical sites, wounds, and other injuries can be treated by reducing edema and increasing circulation in the specific part of the animal 138 treated by the calibrated, compressed section of the garment 100.

The garment 100 is intended for use by both veterinarians and other animal care-givers. Although it can be used to treat any wound or injury, the garment 100 is especially intended for post-surgical use after a laceration or other wound has been sealed or a tumor has been removed. By providing pressure to the care site, the garment 100 helps prevent edema at the care site. This edema may either slow the healing process or even cause the care site to become an infected or necrotic site requiring additional treatment. This prevention of edema is especially necessary in the case of tumor removal, because the removal of the tumor creates an empty space, and fluid will seep into this space by osmosis in the absence of any counter-acting pressure. By preventing edema and its attendant problems such as tissue necrosis, additional surgeries to drain or treat the care site are thereby avoided.

A major advantage of the compression garment 100 is that the level of pressure can be set to a specific level and adjusted as needed. By creating multiple independent sections of the garment 100 that are individually wrapped around the animal 138, including the three sections 102, 104, 106 used in the described embodiment of the garment 100 as shown in FIGS. 4 to 6, different levels of pressure can be applied to different parts of the body. The tensioning straps 112 further enable this pressure level to be set to a specific level and allow it to be easily changed to a different specific level as needed. The present embodiment of the garment 100 has pre-calibrated pressure levels of 30 mm Hg and 50 mm Hg written on the garment. The 30 mm Hg level is the mid-range pre-calibrated compression level. Other embodiments of the garment have a different mid-range pre-calibrated compression level. The 50 mm Hg level is the high pre-calibrated compression level. Other embodiments of the garment have a different high pre-calibrated compression level.

An additional advantage of using the garment 100 to cover a wound or surgical site is it creates a barrier to further self-inflicted injury from the animal 138 scratching itself or otherwise disturbing the care site. The garment 100 can additionally be used to hold other items in place between the garment 100 and animal 138, such as heart monitoring equipment used during an extended observation of the animal 138.

The main part of the described embodiment of the garment 100 that touches the animal 138 is constructed using a blend of cotton, rayon, and spandex. This blend is designed to allow air to reach the care site, which will promote healing. Non-breathable fabrics are therefore not used in any parts of the garment that touch the animal, apart from the straps that are wrapped around the hind legs. Because the garment 100 is breathable, it can be worn continuously for up to two weeks without needing to be removed. The tensioning straps 112 are constructed of nylon webbing. This webbing is designed to remain a constant length despite the tension under which the nylon strap is placed. By remaining a constant length, the strap and its associated letters and dash marks is over time able to retain the original distances between the letters that exist in a new garment 100.

Other embodiments of the garment 100 with additional padding or heating means can be used during surgery to prevent the animal 138 wearing the garment 100 from experiencing a fall in body temperature during general anesthesia. Putting an animal 138 under anesthesia shuts down the thermal regulation system, and body temperature can therefore drop dramatically, which can be life threatening. Current solutions to this problem have focused on using heated surgical tables. However, there are numerous problems with this solution, including occasional burns to the animal 138. The use of a padded or heated compression garment 100 containing a temperature monitor is therefore a useful alternative means for maintaining body temperature during and after surgery.

An additional embodiment of the garment 100 achieves even more uniform compression through the use of PowerNet fabric. Pressure garments made using normal textiles are not designed to withstand continuous wear for weeks at a time. After one to two weeks of continuous wear, such garments lose their structural integrity and ability to provide the same level of compression. This problem is solved by using a medical-grade textile. PowerNet fabric is woven in a flexible, open hexagonal net construction which is designed to apply pressure both vertically and horizontally, resulting in four-way, even compression. It easily recovers its original shape after periods of extreme stress and tension caused by the constant wear. The hexagon unit in PowerNet, when stretched over a body, will open to accommodate airflow, making the fabric breathable. Therefore, there are two advantages to using PowerNet fabric as part of the present invention. First, it aids the even application of pressure across all points of the garment 100. Second, it withstands long-term wear while maintaining its structural integrity and ability to continue applying pressure as evenly and with the same strength as when the garment 100 was first worn.

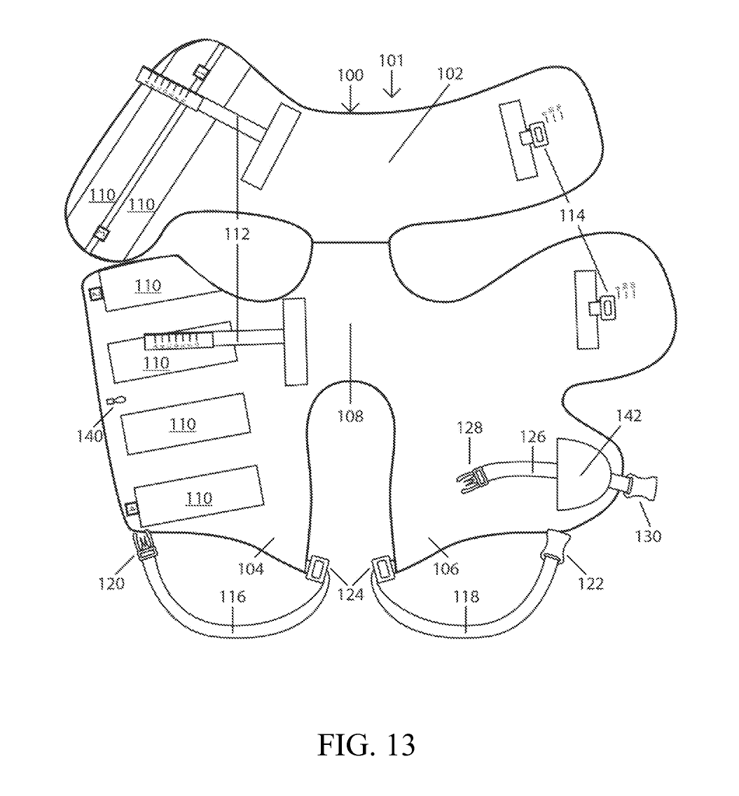

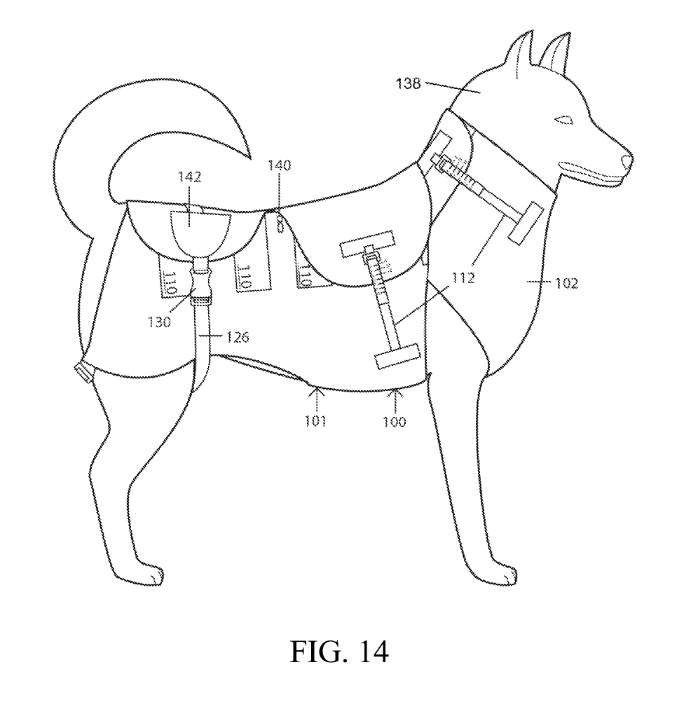

An additional embodiment of the garment 100 is designed to accommodate a Jackson-Pratt drain being used in conjunction with the garment 100. A Jackson-Pratt drain is a closed-suction medical device that is commonly used as a post-operative drain for collecting bodily fluids from surgical sites. It is used to reduce the risk of fluid build-up at a surgical site, to monitor how much fluid is draining from the body, and to keep the surgical site clean. The device consists of an internal drain connected to a grenade-shaped bulb via plastic tubing. As a primary goal of the present invention is to reduce fluid build-up at a care site, the construction of the garment so that a Jackson-Pratt drain can be used in conjunction with it allows for even greater reduction of fluid build-up at care sites. Referring now to FIG. 13, in this embodiment, a nylon loop 140 exists on the left bottom section 104 of the garment 100 through which the drain tubing can be threaded. This inserted tubing will then be placed inside the care site being drained. The portion of the inserted tubing exterior to the garment 100 connects to the bulb portion of a Jackson-Pratt drain. The garment contains a pocket 142 on the right bottom section 106 of the garment 100 designed to house the bulb. With the drain's bulb and tubing thus secured by the garment 100, the drain is well-protected from being disturbed by the animal 138 wearing the garment 100, and the drain can easily be carried around by the garment-wearing animal 138 with little risk that the drain will become dislodged from its proper placement. This embodiment of the garment is placed around the animal 138 using the same steps disclosed in FIGS. 3 to 6. FIG. 14 is a view of this embodiment of the garment 100 placed securely around the animal 138.

While the present invention has been related in terms of the foregoing embodiments, those skilled in the art will recognize that the invention is not limited to the embodiments described. The present invention can be practiced with modification and alteration within the spirit and scope of the foregoing description. Thus, the description is to be regarded as illustrative instead of as restrictive on the present invention.

What is claimed is:

1. A compression garment for use on an animal, the garment having a tri-part structure bisected by a horizontal axis into an elongated top section and two opposing bottom sections, the garment containing fasteners for securing the garment around the animal wearer.

2. The garment of claim 1, wherein the garment has tensioning straps for further tightening the secured garment.

3. The garment of claim 2, wherein the tensioning straps and the parts of the garment next to the secured tensioning straps contain markings for use in securing the straps at set lengths.

4. The garment of claim 3, wherein the tensioning strap markings correspond to pre-calibrated levels of compression, allowing each portion of the garment tightened by a tensioning strap to be set to a specific level of compression.

5. The garment of claim 4, wherein the garment is secured with no discernible level of compression, the marking on the tensioning strap that lines up with a marking on the garment denoting no pressure is noted by a person securing the garment around the animal, and then the tensioning strap is pulled until the same marking on the tensioning strap is in line with another marking on the garment denoting a specific pre-calibrated level of compression, thereby securing the portion of the garment tightened by that tensioning strap with that specific pre-calibrated level of compression.

6. The garment of claim 4, wherein the garment is constructed of PowerNet fabric.

7. The garment of claim 4, wherein one of the opposing bottom sections has a gap in the middle of the side of the section that is distal from the other bottom section, such gap allowing the animal to move through its normal range of motion while wearing the garment.

8. The garment of claim 7, wherein the gap allows the garment to be worn over and around other medical equipment and bandaging attached to the animal.

9. The garment of claim 4, wherein the garment contains an opening for inserting and securing the tubing from a Jackson-Pratt drain and a pocket for holding the bulb of a Jackson-Pratt drain.

10. The garment of claim 9, wherein the garment is constructed of PowerNet fabric.

11. A compression garment for use on an animal which contains an opening for inserting and securing the tubing from a Jackson-Pratt drain and a pocket for holding the bulb of a Jackson-Pratt drain.
